(19) United States
(12) Patent Application Publication
(10) Pub. No.: US 2021/0228465 A1
(43) Pub. Date: Jul. 29, 2021

(54) PERSONAL CARE PRODUCT FORMULATIONS WITH ADJUSTABLE PRODUCT DEPOSITION

(71) Applicant: KDC US Holdings Inc., Lynchburg, VA (US)

(72) Inventor: Sabine Reines, Bromont (CA)

(73) Assignee: KDC US Holdings Inc., Lynchburg, VA (US)

(21) Appl. No.: 17/058,987

(22) PCT Filed: May 29, 2018
(86) PCT No.: PCT/US2018/034923

§ 371 Date: Nov. 25, 2020

(51) Int. Cl. A61K 8/58; A61Q 15/00; A61K 8/34; A61K 8/25; A61K 8/73; A61K 8/37; A61K 8/99; A61K 8/19; A61K 8/60; A61K 8/36; A61K 8/9789; A61K 8/02

(52) U.S. Cl. CPC A61K 8/58; A61Q 15/00; A61K 8/345; A61K 8/25; A61K 8/732; A61K 8/0229; A61K 8/99; A61K 8/19; A61K 8/60; A61K 8/361; A61K 8/9789; A61K 8/375

(57) ABSTRACT

The present invention provides formulations and associated manufacturing methods for solid personal care products in which the amount of product deposition achieved when the product is used by the consumer, a characteristic known as pay-off, can be calibrated by the manufacturer. This is done by varying the levels of several ingredients that comprise the molten form of the product. The amounts of the ingredients propanediol and glycerin, sucrose cocoate, silica, and starch can be adjusted to form a final product with desired pay-off. In accordance with the invention, it is possible to vary one or more, or all five of these ingredients for the final formulation in order to arrive at the preferred pay-off.

### FIELD OF THE INVENTION

The present invention relates to formulations and methods relating to solid personal care products, such as deodorant sticks. The formulations for the products can be calibrated using the features of the invention to achieve varying product deposition according to preference.

### BACKGROUND OF THE INVENTION

There are many forms of solid personal care products which are applied by consumers by gliding the solidified product over the target surface, usually the skin. For example, stick deodorants are typically applied in this manner. Sunblock formulations are also available in stick form. Many cosmetics such as lipstick, lip balm, cover-up, and eye shadows are provided in this product format. Also commonly available are solidified sticks for targeted application of hair coloring, to cover up greying hair or to add color accents.

The desired amount of product to be deposited onto the target surface when applied by the user can vary depending on the product and/or the user's preference. For instance, for lipsticks and lip balms, it would usually be desired to impart a very thin layer of product, as the lips are a sensitive and visible area and over-application of these products will be noticeable. For stick products used as a temporary color for hair, a softer product is generally desired as the hair can accommodate more tinted cosmetic product than most other types of applications. As well, for products such as deodorants and antiperspirants, it would usually be desired to deposit a more significant quantity of product with each application of the stick, so that adequate product is available to provide protection against odor or wetness throughout the day. However, this should be balanced with the need to have the product deposited in a thin enough layer that excess product does not cake or get transferred to clothing.

In the field of personal care products, the amount of product deposited from a solid stick formulation is known as “pay-off”. A softer product tends to have greater pay-off, and a harder product has a lower pay-off. For a deodorant applied to the armpit, on average the amount of product applied per application ranges from 150 mg-200 mg. For deodorants with high pay-off, a higher amount than this range will typically be applied. For deodorants with low pay-off, a lower amount than this range will typically be applied.

Both consumers and manufacturers of solid personal care products have an interest in customizing the level of pay-off for a given product. For example, for solid stick deodorants, many consumers prefer a somewhat harder product which glides well on the skin and which has lower pay-off, whereas others prefer a softer product which leaves a greater amount deposited on the skin. As well, manufacturers have an interest in increasing pay-off so that product is used up faster, while balancing this with consumer preferences such as in regard to the consistency of the stick and the feel of the product.

Given the above, it would be advantageous to have model formulations and formulation techniques for readily and precisely calibrating the amount of pay-off for a solid personal care product, which balances the desired product features of the consumer and the manufacturer, and is also made with the appropriate consistency for achieving the purpose of the product.

### SUMMARY OF THE INVENTION

The present invention provides formulations and associated manufacturing methods for solid personal care products in which the amount of pay-off in the resulting product can be calibrated by the manufacturer. This is done by varying the levels of several ingredients that comprise the molten form of the product. When the solid product is formed following cooling of the heated molten product in a mold, the solid product will have the desired level of pay-off.

In one embodiment of the formulation, the amounts of the base ingredients 1,3-propanediol (hereafter referred to as propanediol) and glycerin can be adjusted to form a final product with desired pay-off. In another embodiment, the amounts of sucrose cocoate can be manipulated with the same general result. In a still further embodiment, the amounts of other commonly used ingredients in personal care formulations, namely silica and starches, can also be strategically adjusted with the same goal of calibrating the product's pay-off.

In accordance with the invention, it is possible to vary one, or all five of the above ingredients for the final formulation in order to arrive at a preferred pay-off.

The present invention is further directed to manufacturing methods for making and forming personal care products in accordance with the described formulations.

The present invention is also directed to a stable and efficacious deodorant stick product manufactured in accordance with the specified method and in accordance with the claimed formulations.

### BRIEF DESCRIPTION OF THE FIGURES

The present invention will now be better understood with reference to the description, the tables contained therein which provide ingredient ranges for tested formulations, and to the accompanying figures in which:

FIG. 1 is a table summarizing the results of efficacy testing of 25 subjects before application of tested product;

FIG. 2 is a table summarizing the results of efficacy testing of the 25 subjects at 8 hours after application;

FIG. 3 is a table summarizing the results of efficacy testing of the 25 subjects at 16 hours after application;

FIG. 4 is a table summarizing the results of efficacy testing of the 25 subjects at 24 hours after application;

FIG. 5 is a table summarizing the averaged results of the efficacy testing for the 25 subjects;

FIG. 6 is a graph demonstrating the efficacy of the invented formulation, in which percent odour reduction over time is shown for the invented formulation versus the control.

### DETAILED DESCRIPTION OF THE PREFERRED EMBODIMENTS

The present invention relates to various formulations for personal care products such as solid stick deodorants, which can be strategically calibrated for hardness according to the desired amount of product deposition for the resulting product when used by the consumer. As also shown, when used to form deodorant sticks, the adjustable pay-off for the resulting products does not interfere with other necessary features for the deodorant, namely product stability and efficacy. Deodorants manufactured in accordance with the formulations and manufacturing methods outlined herein showed 24 hour efficacy in terms of odour reduction, and excellent product stability.

In a first embodiment, the deodorant composition comprises propanediol combined with glycerin. Both are commonly used ingredients for solid personal care products, but what has been discovered is that strategically varying the relative amounts of these ingredients can have a significant impact on the hardness of the final product. Hardness has a directly inverse relationship to pay-off.

Propanediol for the formulations is available from a number of suppliers. When incorporated in a formula for a personal care product, propanediol acts as a humectant, emollient, and a solvent. Glycerin is also a common ingredient in personal care products, and acts mainly as a humectant.

While both propanediol and glycerin are known ingredients for personal care product formulations, it has been discovered that generally, increasing the amount of propanediol and decreasing the amount of glycerin, within certain ranges as described herein, results in a harder resulting product with lower pay-off. This is not a completely linear relationship with respect to glycerin; decreasing the glycerin to under 20% has been seen to result in an increase in softness.

In a further embodiment of the invention, the amount of starch in the formulation may also be manipulated to impact the hardness of the final product. Common starches, such as corn (Zea Mays) starch, are used in personal care formulations to improve the texture and feel of the solid product. A common range for incorporation of starch is 0.5 to 4% by weight. Incorporating starch levels towards the mid-higher end of this range results in softening the resulting solid product, thus increasing the payoff.

Silica is also used in personal care formulations, typically to act as a thickener and to enhance viscosity. A typical range for silica is 0.5-4%. It has been found that increasing silica towards the mid-higher end of this range results in a softer product with greater payoff.

Sucrose cocoate is an ingredient for personal care formulation, which is based on the sucrose esters of coconut acid. It may be used as a clarifying agent, and also acts as a hydrophilic emollient. In one embodiment, the sucrose cocoate may optionally be incorporated at a level of up to 2% by weight, based on the total weight of the composition, in a formulation for solid stick deodorants. If the formulation contains very little or no sucrose cocoate, the resulting stick will tend to have a lower pay-off. At higher levels approaching 2%, the stick will have a higher pay-off. Amounts of sucrose cocoate between these extremes can be successfully used in formulating personal care product sticks, with a particularly preferred range being 0.5-1.2% by weight.

As will be understood from the description provided herein, in one embodiment the invention provides a personal care product formulation comprising by weight, based on the total weight of the composition: from about 31-45% propanediol, from about 8-25% glycerin, from about 0.5-4% silica, and from about 0.5-4% corn starch. It will be understood that the use of the term “about” in relation to a range applies to both ends of the range provided.

In another embodiment, the formulation is a deodorant formulation, and further comprises as an optional ingredient, sucrose cocoate for further conditioning and softening properties. The amount of sucrose cocoate can range from 0.05-2%.

In another embodiment, a deodorant formulation is provided that comprises, by weight based on total weight of the composition, from about 31-45% propanediol, from about 8-25% glycerin, from about 0.5-4% silica, from about 0.5-4% corn starch, from about 0.1-1% grapefruit seed extract, from about 6-12% sodium stearate, from about 0.5-2% zinc ricinoleate, from about 0.5-2% polyglyceryl-3 caprate, from about 0.1-1% Saccharomyces ferment filtrate, from about 0.05-1.5% fragrance and natural plant essences, from about 0.05-0.1% sodium bicarbonate, water q.s., and essential oils q.s.

Zinc ricinoleate acts as an effective odour neutralizer. The Saccharomyces ferment used is a product of fermentation that is beneficial for inclusion in a deodorant as it fights the creation of malodor by converting ammonia into amino acids. Ready-made Saccharomyces ferment products are available from a number of suppliers to the personal care products industry.

In a further embodiment, a formulation for a personal care product having high pay-off is provided that comprises, by weight based on total weight of the composition, a combination of one or more of the following ingredients: propanediol in the amount of 31-33%, glycerin in the amount of 21-26%, silica in the amount of 2.3-2.5%, corn starch in the amount of 2.3-2.5%, and sucrose cocoate in the amount of 0.9-2%.

An alternative embodiment for a personal care product having high pay-off is provided that comprises, by weight based on total weight of the composition, a combination of one or more of the following ingredients: propanediol in the amount of 31-41%, glycerin in the amount of 8-20%; silica in the amount of 2.0-2.5%; corn starch in the amount of 1.0-2.5%, and sucrose cocoate in the amount of 0.5-1.2%.

In yet a further embodiment, a formulation for a personal care product having low pay-off is provided that comprises, by weight based on total weight of the composition, a combination of one or more of the following ingredients: propanediol in the amount of 35-45%, glycerin in the amount of 8-22.9%, silica in the amount of 0.5-2.2%, and corn starch in the amount of 0.5-2.2%.

### Testing Product Hardness

As referred to above, the hardness of a product is inversely proportional to pay-off. For solid cosmetics and personal care products, product hardness may be measured using a standard methodology which measures penetration of the product within a given time frame by a needle installed in a penetrometer. There are some variations of the method in use in the field. In the testing method used by the inventor, the first step was to incubate the test products in a water bath for at least 24 hours, until the products reached 26° C., in order to normalize the product temperatures prior to measurement. 26° C. is considered an appropriate test temperature as it is at the higher end of the typical range for room temperatures which reflects the conditions in which the product would be used.

A penetrometer (PRECISION UNIVERSAL™) fitted with a standard needle probe conforming to ASTM specification D-1321, and a 2.5 g weight, was then aligned with the surface of the product, and released in order to allow penetration of the needle. At precisely 5 seconds following release, the depth of penetration was measured in units of a tenth of a mm (100 μm). A higher numerical result means that the product was softer, being more readily penetrated, and therefore has a higher pay-off.

It has been found that a target range of 100 to 240 μm results in a good level of hardness and pay-off for deodorant and antiperspirant sticks in particular. Testing results close to 100 μm indicate that the product has lower payoff and is suitable for those consumers who prefer a very thin layer of product being deposited onto the skin. Testing results close to 240 μm indicate that the product has higher payoff, and is suitable for those consumers who prefer a creamier, softer consistency for their stick product.

### Experimental Formulations

Sample deodorant products were formulated with varying amounts of the test ingredients combined with standard amounts of the other ingredients required for a solid deodorant formulation (such as sodium stearate, water q.s.) and then tested with the penetrometer in accordance with the methodology described above. All samples were aged to 12 weeks prior to testing to ensure that the product had sufficiently stabilized in its solid form prior to measurement. For each test, four measurements of penetration were taken, and the numbers at the upper and lower extremes were discarded as possible outliers. The remaining numbers were averaged to arrive at the final results.

First, a comparison was done between two base formulations, Formulas SM1 and SM2, to determine the effect of increases in corn starch and silica. The ingredients in these formulas were as follows:

TABLE 1
Effect of increased silica and starch

| | % w/w - | % w/w - |
|---|---|---|
| Ingredient | Formula SM1 | Formula SM2 |
| Propanediol | 31.00 | 31.00 |
| Glycerin | 26.00 | 26.00 |
| Silica | 2.50 | 2.00 |
| Corn starch | 2.50 | 2.00 |
| Other ingredients | q.s. | q.s. |
| Penetrometer Measurement | 215 μm | 192 μm |

As mentioned above, “other ingredients” consists of at least sodium stearate and water, and were kept consistent between all compared formulations. Where quantities of ingredients differ between the compared formulations, additional water q.s. was used to make up the difference. Additional ingredients such as polyglyceryl-3-caprate, allantoin, grapefruit seed extract, zinc ricinoleate, Saccharomyces ferment, were also used, but have been found to not impact product hardness. Fragrances and essences were not included in these test formulations, which were all unscented in order to focus only on the effects of varying target ingredients.

As shown above, formula SM1 contained 2.50% of each of corn starch and silica, whereas formula SM2 contained 2.00% of each of these ingredients. In other respects, these formulations were identical. To compensate for the different amounts of these ingredients, increased water q.s. was used.

The values obtained through averaged penetrometer testing were 215 and 192 respectively. It can be seen with this test that an increase of silica and starch correlates to softer product with higher pay-off. In this test, the difference between hardness of the two formulations was approximately 11%.

Increasing the amounts of silica and starch is believed to have resulted in an increase in opportunities for foaming and for air bubbles to be ultimately incorporated into the stick structure, resulting in a softer, more voluminous composition for the stick.

In a further trial, formula SM2 was compared to formula SM2A, which contained an increased amount of propanediol and a decreased amount of glycerin.

TABLE 2
Effect of increased propanediol and decreased glycerin

| | % w/w - | % w/w - |
|---|---|---|
| Ingredient | Formula SM2 | Formula SM2A |
| Propanediol | 31.00 | 35.00 |
| Glycerin | 26.00 | 22.3 |
| Silica | 2.00 | 2.00 |
| Corn starch | 2.00 | 2.00 |
| Other ingredients | q.s. | q.s. |
| Penetrometer Measurement | 192 μm | 77 μm |

As compared to the original Formulation SM2 containing 31% propanediol and 26% glycerin, the changes in levels of these ingredients caused the base to become significantly harder. This testing reveals that an increase in propanediol combined with a corresponding decrease in glycerin within these ranges, results in a harder product with less pay-off.

These changes likely occurred because propanediol is a highly effective solvent, and increasing this ingredient may result in better dissolution of other ingredients in the formulation. Glycerin, on the other hand, is a humectant with a sticky, syrup-like texture. It tends to have a softening effect when incorporated at higher levels in solid personal care formulations.

Interestingly, increasing propanediol and decreasing glycerin do not always result in a harder final product. A further trial was done with further changes to the amounts of these ingredients.

TABLE 3
Effects of further increased propanediol and decreased glycerin

| | % w/w - | % w/w - | % w/w - |
|---|---|---|---|
| | Formula | Formula | Formula |
| Ingredient | SM2 | SM2A | SM2B |
| Propanediol | 31.00 | 35.00 | 41.00 |
| Glycerin | 26.00 | 22.3 | 17.3 |
| Silica | 2.00 | 2.00 | 2.00 |
| Corn starch | 2.00 | 2.00 | 2.00 |
| Other ingredients | q.s. | q.s. | q.s. |
| Penetrometer Measurement | 192 μm | 77 μm | 110 μm |

The experiment with Formula SM2B as shown in Table 3 revealed that there is not a linear relationship between increases in propanediol to increases in hardness. Increasing propanediol to 41% and decreasing glycerin to 17.3% resulted in penetrometer readings of 110 μm, whereas the prior increased level of propanediol and decreased level of glycerin resulted in readings of 77 μm. Reducing glycerin by such a significant amount to under 20% resulted in softer sticks. This phenomenon is believed to be founded in the observation that the decrease in glycerin by such an extent results in a lower solidification point for the stick. In other words, the stick solidifies at a cooler temperature than usual. This means that when the stick is subjected to the standard testing procedure, which requires bringing the stick up to the test temperature of 26° C., it will tend to be softer.

A further trial was done to test the impact of sucrose cocoate addition. To Formulation SM2A, sucrose cocoate at 0.9% by weight was added. Penetrometer measurements resulted in an average reading of 102 μm. This confirmed that the addition of sucrose cocoate in the amount indicated resulted in a softer product.

TABLE 4
Effect of sucrose cocoate addition

| | % w/w - | % w/w - |
|---|---|---|
| Ingredient | Formula SM2A | Formula SM2A1 |
| Propanediol | 35.00 | 35.00 |
| Glycerin | 22.3 | 22.3 |
| Sucrose cocoate | 0.00 | 0.90 |
| Silica | 2.00 | 2.00 |
| Corn starch | 2.00 | 2.00 |
| Other ingredients | q.s. | q.s. |
| Penetrometer Measurement | 77 μm | 102 μm |

The above results provide guidance in terms of ranges for ingredients that can be varied to control the hardness, and ultimately, the pay-off of the resulting product. One can vary some or all five of the ingredients—propanediol, glycerin, corn starch, silica, and sucrose cocoate. Changes in the amounts of propanediol and glycerin provide for the greatest impact on product hardness, but the amounts of the other ingredients can also be manipulated to achieve further changes in consistency.

In accordance with the invention, it is beneficial to have the choice of multiple parameters to control pay-off in the resulting product. As is known to the skilled formulator in this field, along with pay-off, it is important to monitor impacts of changes in ingredients on other features impacting the final product, such as product feel, coloring, and general appearance. Changes in one or more of the above listed ingredients may compromise other important product features, so it is often preferred to make smaller changes in a number of ingredients so that overall product quality is not compromised at the expense of pay-off.

A summary of the results from the testing of different formulations is shown below in Table 5.

TABLE 5
Effects of Ingredient Changes

| Ingredient Change | Hardness | Pay-off |
|---|---|---|
| Increased Starch; Increased Silica | Decrease | Increase |
| Increased Propanediol; Decreased Glycerin | Increase | Decrease |
| (within described ranges) | | |
| Increased Sucrose Cocoate | Decrease | Increase |

A sample formulation of the invention which included other ingredients typical for a natural deodorant formulation was prepared, and details are shown as Formula C1 below. This formulation had high pay-off, achieving an averaged value of 205 μm in penetration testing. This formula also displayed excellent efficacy and stability.

TABLE 6
Formula C1

| Material | % (w/w) |
|---|---|
| Propanediol | 40.0% |
| Glycerin | 10.0% |
| Sodium Stearate | 8.0% |
| Zinc ricinoleate | 2.0% |
| Polyglyceryl-3-caprate | 0.99% |
| Silica | 2.0% |
| Grapefruit Seed Extract | 0.5% |
| Sucrose Cocoate | 0.9% |
| Corn (zea mays) starch | 1.0% |
| Saccharomyces ferment filtrate | 0.50% |
| Calendula Officinalis flower extract, | 0.40% |
| Aloe Barbadensis leaf juice, tocopherol, | |
| glycine soybean oil, allantoin | |
| Water | q.s. |
| | 100.0% |

The above Formula C1 was also prepared with no sucrose cocoate, to provide for a low pay-off formulation. Without sucrose cocoate, the averaged penetration level measured during testing was 155 μm, further confirming the earlier testing results in respect of the impact of including sucrose cocoate.
Another version of Formula C1 was also prepared with the addition of 0.05% sodium bicarbonate, an additional deodorizing active. Inclusion of sodium bicarbonate did not impact pay-off.

### Manufacturing Method

The manufacturing method for making sticks using the formulations of the invention is now provided. While the general manufacturing process for creating deodorant stick products is known, there are certain specific steps, temperatures, and orders of ingredient addition that are necessary when working with the described formulations. It is also necessary to make certain pre-mixes of selected ingredients to maximize dissolution and distribution in the final product.

First, two premixes of ingredient are made and set aside. In premix A, a portion of the total propanediol being used for the final product, anywhere between one third to one half, is mixed with polyglyceryl-3-caprate and zinc ricinoleate while agitating and heating to a relatively high temperature, with a range between 80-95° C. being acceptable for mixing and homogenization purposes. Once visibly uniform, premix A is set aside and maintained at this temperature.

In premix B, a further portion of the total propanediol, approximately 10-25%, is mixed with water q.s., and cornstarch at ambient temperature. When uniform, the premix is also set aside.

Next, the remaining amount of propanediol, glycerin, allantoin (if used), and sodium bicarbonate (if used) is mixed under agitation at an elevated temperature of between 80-95° C. The sodium stearate is then added while maintaining agitation and temperature in the same temperature range. Premix A is then added under agitation.

At this point, the silica is added and the speed of agitation is reduced. The mixture is stirred while maintaining the temperature in the same temperature range until the silica has been incorporated.

The batch is then permitted to cool to a mid-temperature range, approximately 60-75° C., while remaining under agitation, and Premix B is incorporated. Further ingredients are then added once the target temperature range has been reached: Sucrose cocoate (if used), grapefruit seed extract, Saccharomyces ferment extract are then added, along with any remaining volatile fragrances, essences, and oils. Mixing continues in this temperature range for at least 20 minutes, and up to an hour, to ensure homogenization. The resulting emulsion is then used to fill standard deodorant packaging, and the product cooled to form stick deodorants. The solidified sticks are generally stored at room temperature for several weeks to allow the product to totally set and stabilize prior to shipment.

### Efficacy Testing

To confirm efficacy, formula C1 was tested and displayed excellent efficacy. In the industry, efficacy testing for deodorants is typically conducted by way of panel studies, in which the test substances and control substances are applied to the axilla of human subjects, and odour is then assessed at intervals by an objective panel of reviewers. This type of deodorant testing is referred to in the industry as a “sniff test”. Such a sniff test was conducted in this case, involving twenty-five subjects and three judges. Each subject's two armpits were tested using one as a control (which was untreated), and one having application of the invented deodorant formulation. The testing conformed with the standard specified in ASTM International Standard E1207-09, being randomized and single-blinded; judges were blinded to the identity of the control and treated armpits. Uniform amounts of deodorant were applied by technicians in each treated armpit; approximately 200 mg for men and 150 mg for women.

As seen in Table 7, the twenty-five subjects were tested using a 10-point subjective axillary odour scale, with a ranking of 0 indicating no odour, and a ranking of 10 indicating a very strong sweaty odour. Middle rankings of 4 to 6 indicated definitely perceptible to moderate odours.

TABLE 7
Efficacy Panel Test - 10 Point Armpit
Odour Intensity Grading Scale

| 0 | 2 | 4 | 6 | 8 | 10 |
|---|---|---|---|---|---|
| Odour absent | Odour barely | Odour | Odour | Odour | Odour |
| | perceptible | definitely | moderate | strong | very |
| | | perceptible | | | strong |

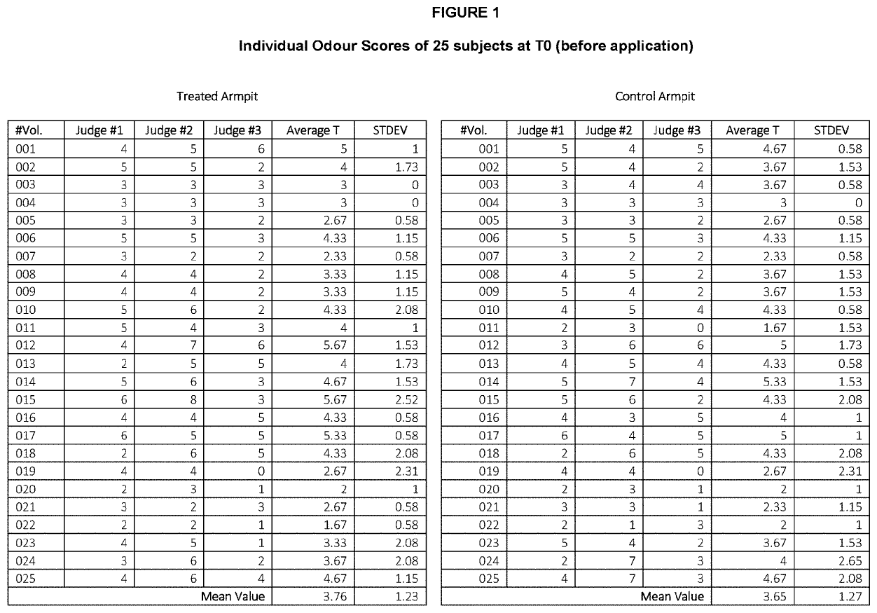

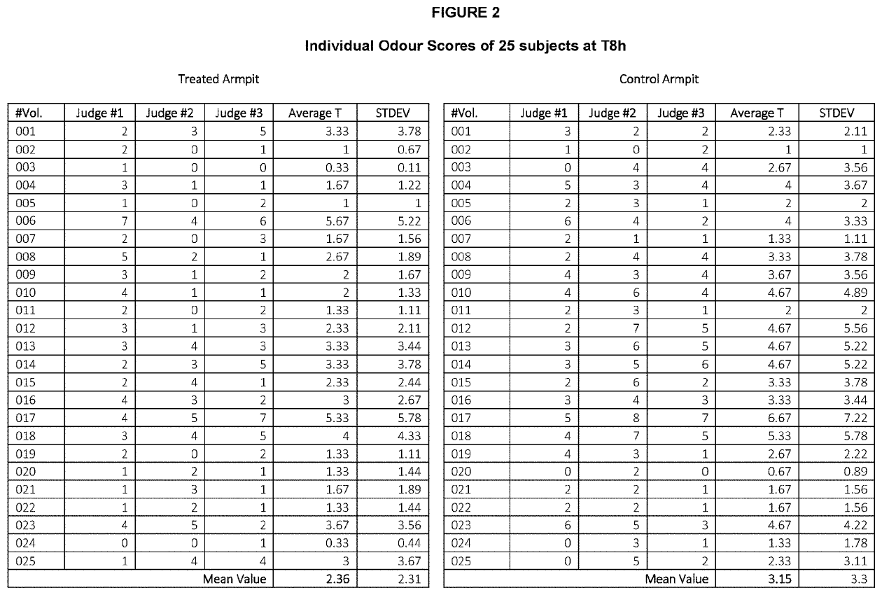

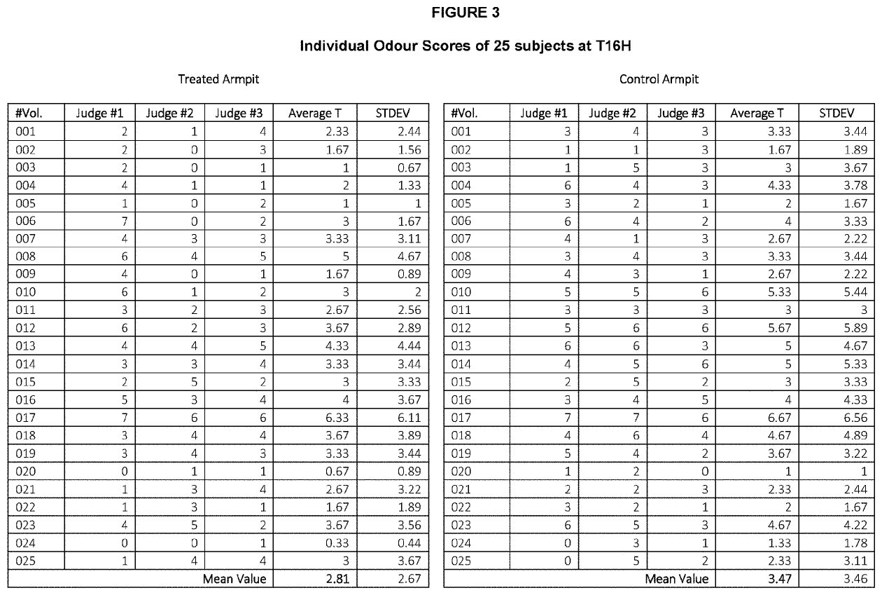

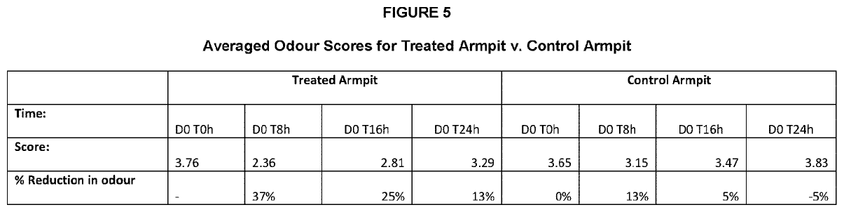

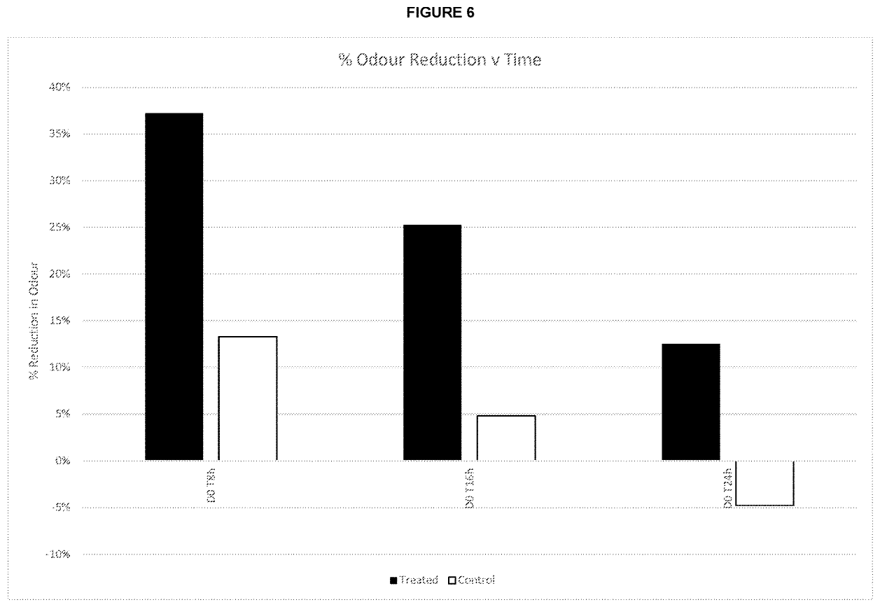

As set out respectively in FIGS. 1-4, subjects were tested prior to treatment (Day 0-T0), and at intervals of Day 0-8 hours, Day 0-16 hours, and Day 1-24 hours post-treatment. The mean results for the twenty-five subjects are presented in FIG. 5. The values demonstrate that the deodorant formulation of the invention was effective at neutralizing axillary odour. As shown in the graph at FIG. 6, at each of 8, 16, and 24 hours post-treatment, the percent odour reduction was significant, ranging from 12 to 37% odour reduction. The differences between treated and control armpits of the subjects were found to be statistically significant.

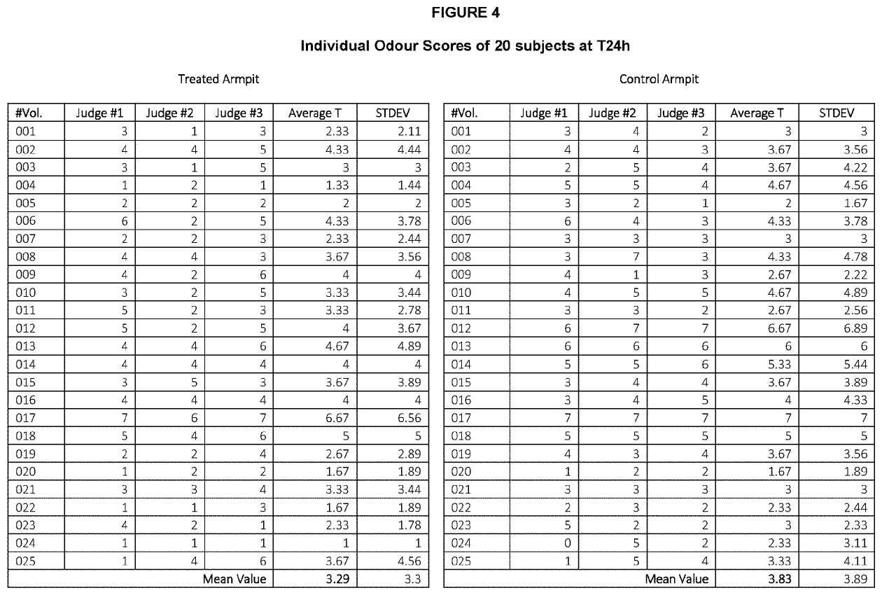

The panel testing results summarized in FIG. 4 show 24 hour efficacy for the deodorant formulation of the invention. This level of odour reduction is excellent and long-lasting, and is in line with the highest industry standards achieved for deodorants formulated primarily from natural ingredients.

Stability testing of formula C1 also confirmed excellent stability following formation of solid sticks. Standard visual testing for color and appearance was performed at the intervals of 0 weeks, 4 weeks, 8 weeks, and 13 weeks. Weight loss testing over time also at temperatures of 5° C., Room Temperature, 30° C., 40° C., and 45° C. produced standard acceptable stability results.

As set out previously, a person of skill in the art may make variations of the formulations based on the variety of ingredients available in the prior art, as described above. It should further be noted that when an amount, concentration or other parameter is given as a range or a list of upper and lower preferable values, it is to be understood as disclosing all ranges formed from any pair of any upper and lower limit. Where a range of numerical values is recited, unless otherwise stated, the range includes the endpoints thereof.

While the invention has been described with reference to specific embodiments thereof, it will be appreciated that numerous variations, modifications, and embodiments are possible to the skilled person. All such variations, modifications and embodiments are to be regarded as being within the spirit and scope of the invention.

What is claimed is the following:

1. A composition for a deodorant, comprising by weight, based on the total weight of the composition: from about 31-45% propanediol; from about 8-25% glycerin; from about 0.5-4% silica; from about 0.5-4% corn starch; from about 0.1-1% grapefruit seed extract; from about 6-12% sodium stearate; from about 0.5-2% zinc ricinoleate; from about 0.5-2% polyglyceryl-3-caprate; from about 0.1-1% Saccharomyces ferment filtrate; and from about 0.05-1.5% fragrance and plant essences.

2. A composition according to claim 1, further comprising from about 0.05-0.1% sodium bicarbonate.

3. A composition for a deodorant, comprising by weight, based on the total weight of the composition: from about 31-45% of propanediol; from about 8-25% glycerin; from about 0.5-4% silica; and from about 0.5-4% corn starch, wherein a penetration test in compliance with ASTM D-1321 results in penetration in the range of 100 to 240 μm.

4. The composition according to claim 3, further comprising sucrose cocoate in the amount of 0.05 to 2%.

5. A composition for a deodorant, comprising by weight, based on the total weight of the composition: from about 31-41% of propanediol; from about 8-20% glycerin; from about 2.0-2.5% silica; from about 1.0-2.5% corn starch; from about 0.5-1.2% sucrose cocoate; wherein a penetration test in compliance with ASTM D-1321 results in penetration in the range of 170 to 240 μm.

6. The composition of claim 1, wherein:
the propanediol is provided in the amount of about 35-45%;
the glycerin is provided in the amount of about 8-22.9%;
the silica is provided in the amount of about 0.5-2.2%;
the corn starch is provided in the amount of about 0.5-2.2%;
and wherein a penetration test in compliance with ASTM D-1321 results in penetration in the range of 100 to 169 μm.

7. The composition according to claim 6, further comprising sucrose cocoate in the amount of about 0.5-1.2%.

8. The composition of claim 6, wherein:
the propanediol is provided in an amount of about 40.0%;
the glycerin is provided in an amount of about 10.0%;
the sodium stearate is provided in an amount of about 8.0%;
the zinc ricinoleate is provided in an amount of about 2.0%;
the polyglyceryl-3-caprate is provided in an amount of about 0.99%;
the silica is provided in an amount of about 2.0%;
the corn starch is provided in the amount of about 1.0%;
the grapefruit seed extract is provided in an amount of about 0.5%;
the Saccharomyces ferment filtrate is provided in an amount of about 0.5%; and
which further comprises sucrose cocoate in the amount of about 0.9%.

9. The composition according to claim 8, further comprising sodium bicarbonate in the amount of about 0.05%.

10. The composition of claim 8, wherein a penetration test in compliance with ASTM D-1321 results in penetration of about 205 μm.

11. A deodorant stick product comprising the composition of claim 8 and a deodorant container, wherein said molten composition is provided in a molten form, poured into a deodorant container, and cooled.

12. A method of manufacturing a deodorant of any one of claims 1-9 comprising the steps of
making a first premix comprised of one half to one third of the propanediol, all of the polyglyceryl-3-caprate, and all of the zinc ricinoleate;
mixing said first premix while heating to a temperature between 80-95° C.;
making a second premix comprised of 10-25% of the propanediol, water q.s., and all of the cornstarch, and mixing until visually uniform in appearance;
Separately from said first premix and said second premix, mixing the balance of the propanediol and all of the glycerin under agitation at 80-95° C. to form a mixture C, Adding sodium stearate to the mixture C;
Adding the first premix to the mixture C;
Adding the silica to the mixture C;
Cooling the mixture C to 60-75° C. under continual agitation;
Adding the second premix to the mixture C;
Adding grapefruit seed extract, Saccharomyces ferment, and volatile fragrances and essences, and oils to the mixture C to form mixture D;
Stirring the mixture D for 20-60 minutes at 60-75° C.; and
Pouring suitable amounts of the mixture D into a deodorant container; and
Cooling said container and mixture D.

13. The method of claim 12, wherein allantoin is also added at the same time as the glycerin.

14. The method of claim 12, wherein sucrose cocoate is added at the same time as the grapefruit seed extract.
